(19) United States
(12) Patent Application Publication
(10) Pub. No.: US 2003/0162165 A1
(43) Pub. Date: Aug. 28, 2003

(54) Chemical signal enhancement of dynamic intensity-based intracellular protein-and fluorophore-based redistribution assays for drug-screening

(76) Inventors: Per O.G. Arkhammer, Helsingborg (SE); Morten Heide, Holte (DK); Ole Thastrup, Birkerod (DK); Grith Hagel, Dragor (DK); Kurt Marshall Scudder, Virum (DK); Morten Praestegaard, Ballerup (DK)

Correspondence Address: BIRCH STEWART KOLASCH & BIRCH, PO BOX 747, FALLS CHURCH, VA, 22040-0747, US

(21) Appl. No.: 10/258,668

(22) PCT Filed: Apr. 26, 2001
(86) PCT No.: PCT/DK01/00279

(51) Int. Cl. C12Q 1/00; C12Q 1/66

(52) U.S. Cl. 435/4; 435/8

(57) ABSTRACT

The present invention involves that adding certain dyes to the solution outside cells expressing a GFP-tagged proteins in a redistribution assay, enhance the signal. Examples of such dyes are Trypan Blue and Acid Red. This enables redistribution assays to be measured as a change in light intensity. Redistribution movement from the cytosol to the membrane, or vice versa, are illustrated. The apparatus used for measuring the redistribution is an ordinary plate reader or a FLIPRTM type instrument. It is a common feature among the dyes described that the enhancer compound has an absorption spectrum overlapping the emission spectrum of the GFP.

### BACKGROUND

At present, the drug industry is faced with a challenge when it comes to making use of all the information emanating from worldwide genomic projects. Besides information on potential new cell surface receptors that can be used to set up a more traditional panel of assays for drug screening, a multitude of new potential drug targets are being presented. Many of these new proteins are a) intracellular, b) part of an intracellular signalling pathway, c) dependent on both their enzymatic activities (such as kinase, phosphatase or phosphodiesterase activity) and their proper intracellular localization for their impact on the signalling pathway, d) dependent on one or several simultaneous interactions with scaffold or anchor proteins (those that serve to bind and hold the signalling protein in its proper place to enable its transmission of signal downstream) and downstream effector proteins that receive the “message” and transduce it further or change their activity to affect cellular function directly, e) redistribute within the cell from one location where they receive their input/activation signal to another one where they deliver their output signal. An intracellular signalling pathway involving several transduction steps, redistribution of some signalling components, and several anchoring or scaffolding proteins thus presents a multitude of potential targets for drug modulation, targets that are based on protein-protein interactions inside cells.

The problem for the drug industry is to invent and develop assay systems that reliably can utilise these events for screening of chemical compounds to find new entities that can be developed into drugs. Such assays have one fundamental characteristic: they require living cells that present the above-described signalling system in a functionally competent form, such that the protein-protein interactions can be observed as they occur as part of the signalling process. One approach to measuring protein-protein interactions inside living cells is to mark or tag the intracellular protein one wants to study with a fluorophore (a chemical compound or a fluorescent protein such as GFP). The protein of interest is preferably one that redistributes upon activation. One can then follow the movements of the tagged protein within the cell using a technique such as fluorescence microscopic imaging. In order to be useful as a method for drug screening, the measurement of the redistribution must be quantifiable in a reproducible manner. One way to accomplish this is to record images of the redistribution phenomenon, and mathematically processes the images to extract the desired information. For example, one might measure the change in light intensity in a particular subcellular compartment as the fluorescently tagged protein redistributes into or out of that compartment. Such a measurement might require taking multiple images during a fast dynamic process, or might only require a single image taken at the endpoint or a steady state point during a slower redistribution. WO00/17624 and WO97/45730 describe the single-time-point application of this concept. WO98/45704 describes dynamic application of this concept.

One limitation of the methods described above is that they are slow compared to other more commonly used assay methods. A de facto requirement of screening as it is currently practised is that the throughput (the number of test compounds that can be assayed in a given amount of time) must be sufficient to get through a typical collection of compounds in a reasonable time. Acceptable throughput is several hundred to several thousand samples per day. This has primarily been achieved by parallelization, i.e., making instruments that perform many individual measurements simultaneously, and by automation. While automated microscopes have been described (see WO00/17643 and WO00/03246) and offered commercially, there is currently no method for parallelizing such measurements.

Modulation of the interaction between fluorescently tagged proteins may also be detected using FRET (fluorescence resonance energy transfer) (Mol. Endocrinol. 12(9) (1998); P. B. Fernandes Curr. Opin. Chem. Biol. 2(5) (1998) Review), FP (fluorescence polarization) (M. Jolley J. Biomol. Scr. 1 (1996); P. B. Femandes Curr. Opin. Chem. Biol. 2(5) (1998) Review) or FCS (fluorescence correlation spectroscopy) (R. Riegler J. Biotechnol. 41 (1995); P. B. Fernandes Curr. Opin. Chem. Biol. 2(5) (1998)). Each of these methods can be (and in some cases has been) applied to the measurement of protein-protein interactions in vitro. However, none of these techniques has been successfully applied to drug screening against intracellular protein targets in intact cells except through the use of advanced microscopic techniques. Therefore, these assay types are severely limited with regard to speed, and thus incapable of the throughput required for screening techniques in drug discovery.

Another requirement for assays used in drug screening is adequate precision, where precision is a quantitative measure of the ability to discern between those compounds which cause a response in an assay and those that don't. The precision of an assay is usually expressed as the Coefficient of Variation, or CV (CV=(assay standard deviation/assay range)×100), expressed as a percentage. A smaller CV indicates higher precision. Most screening scientists would hesitate to implement an assay with a CV greater than about five percent. It is possible to mitigate a high CV in some assays by running multiple replicates of the each sample and averaging the results. However, one rapidly reaches a point of diminishing returns: the increase in time required to run the assay is essentially linear with the number of replicates, but the increase in precision with each additional replicate rapidly becomes negligible. Consequently, few screening scientists would consider implementing an assay that required more than eight to ten replicates to achieve an overall CV of five percent. To run this argument in reverse, anything that reduces the CV of a measurement directly impacts the throughput of the assay by reducing the number of replicates one must run to achieve statistical significance of the results. An assay with high precision (low CV) represents the best possible scenario: one need only assay each test compound in singlicate, and one can detect weaker positive responses out of the normal background noise of the assay. Furthermore, it gives the possibility to change the format of the assay to one in which more samples can be measured simultaneously, which would also increase the throughput. Certain compounds can affect the measured fluorescence. The fact that the dye Trypan Blue can be used to reduce green fluorescence from compounds such as fluorescein is well known (S. Sahlin, J.Hed, I. Rundquist. J. Immunol. Meth. 60, 115 (1983); Y. Hansson, et al. J. Immunol. Meth. 100, 261 (1987); C. P. Wan, C. S. Park, B. H. Lau. J. Immunol. Meth. 162, 1 (1993)). It has also found commercial use in assays (Vybrant™ Phagocytosis Assay Kit from Molecular Probes Inc.). The utility of Trypan Blue is that it is excluded from living cells, and thereby can reduce extracellular but not intracellular fluorescence from fluorescein or other green fluorescent compounds. This has been used, for instance, in phagocytosis assays where cells take up fluorescently labelled material from the surrounding medium (S. Sahlin, J.Hed, I. Rundquist. J. Immunol. Meth. 60, 115 (1983); C. P. Wan, C. S. Park, B. H. Lau. J. Immunol. Meth. 162, 1 (1993)). Trypan Blue is added to remove fluorescence from all extracellular label, including label on the outside of the outer leaflet of the plasma membrane, but not from the intracellularly located label. Presumably, the plasma membrane, which is about 70-100 Å thick, is enough of a barrier for the dye not to affect the fluorescence of labelled material that has been ingested by the cell.

Any technique, therefore, which allows the measurement of subcellular protein redistribution to be parallelized in addition to being automated, would strongly impact the development of such assays in a positive manner. By the same token, any method that increases the inherent precision of such measurements enhances their utility, by increasing the throughput, allowing weaker responses to be distinguished from the background noise of the procedure, and offering the possibility of increasing the assay throughput through increasing the density of the assay format.

### SUMMARY OF THE INVENTION

The present invention involves the unexpected finding that some chemical substances, added to the solution outside cells expressing a GFP-tagged protein in a redistribution assay, enhance the signal component of the redistribution response while only causing a marginal increase in assay background and cell-free plate background (see FIGS. 7 and 8). One such compound is Trypan Blue (CAS No. 72-57-1). Despite the fact that Trypan Blue is outside the cells, it reduces the fluorescence from GFP-tagged protein aggregated at the inner face of the plasma membrane resulting in an enhanced signal change as the protein redistributes from the cytosol to the membrane (see FIG. 6, type 1, decrease in signal) or from the membrane to the cytosol (see FIG. 6, type 3, increase in signal). At the same time it is clear that in a redistribution assay that does not involve a plasma membrane aggregation of GFP-tagged protein, such as type 2 in FIG. 6, Trypan Blue has no enhancing effect.

After evaluating several other chemical compounds for a similar enhancing effect, the compound Acid Red 88 (CAS No. 1658-56-6) was identified (see FIGS. 2-4). Acid Red 88 is water soluble but more lipophilic than Trypan Blue, and probably enters the cells to some extent and in a concentration-dependent manner. Thus Acid Red 88 enhances the signal component of the redistribution response in type 1, 2 and 3 redistribution assays.

### DETAILED DISCLOSURE OF THE INVENTION

One solution to the industry's demand for high throughput is the unexpected finding summarised in the following, and detailed described in patent application (WO00/23615) that under certain conditions, some fluorescence plate readers can be used to detect redistribution of fluorescently tagged intracellular proteins as a change in light intensity, i.e. without mathematical processing of images, in ordinary microtiter plates, 96 parallel experiments simultaneously.

In one of its broadest aspects, the invention relates to an improved method, with higher throughput compared to previous methods, for extracting quantitative information relating to an influence on a cellular response, the method comprising recording variation, caused by the influence on mechanically intact living cells, in spatially distributed light emitted from a luminophore, the luminophore being present in the cells and being capable of being redistributed in a manner which is related with the degree of the influence, and/or of being modulated by a component which is capable of being redistributed in a manner which is related to the degree of the Influence, the association resulting in a modulation of the luminescence characteristics of the luminophore, detecting and recording the variation in spatially distributed light from the luminophore as a change in fluorescence intensity using an instrument designed to measure changes in fluorescence intensity, and processing the recorded variation in the spatially distributed light to provide quantitative information correlating the spatial distribution or change in the spatial distribution to the degree of the influence. This is performed by measuring change in light intensity.

The present screening assays have the distinct advantage over other screening assays, e.g., receptor binding assays, enzymatic assays, and reporter gene assays, in providing a system in which biologically active substances with completely novel modes of action, e.g. inhibition or promotion of redistribution/translocation of a biologically active polypeptide as a way of regulating its action rather than inhibition/activation of enzymatic activity, can be identified in a way that insures very high selectivity to the particular isoform of the biologically active polypeptide and further development of compound selectivity versus other isoforms of the same biologically active polypeptide or other components of the same signalling pathway.

In one embodiment of the invention, the recording of variation in light intensity is made at a single point in time after the application of the influence. In another embodiment, the recording is made at two points in time, one point being before, and the other point being after the application of the influence. The result or variation is determined from the change in fluorescence compared to the fluorescence measured prior to the influence or modulation. In another embodiment of the invention, the recording is performed at a series of points in time, in which the application of the influence occurs at some time after the first time point in the series of recordings, the recording being performed, e.g., with a predetermined time spacing of from 0.1 seconds to 1 hour, preferably from 1 to 60 seconds, more preferably from 1 to 30 seconds, in particular from 1 to 10 seconds, over a time span of from 1 second to 12 hours, such as from 10 seconds to 12 hours, e.g., from 10 seconds to one hour, such as from 60 seconds to 30 minutes or 20 minutes. The result or variation is determined from the change in fluorescence over time. The result or variation could also be determined as a change in the spatial distribution of the fluorescence over time.

The recording of light intensity can be done with various types of apparatuses known to the person skilled in the art. Typically such apparatus comprises the following components: (a) a light source, (b) a method for selecting the wavelength(s) of light from the source which will excite the luminescence of the luminophore, (c) a device which can rapidly block or pass the excitation light into the rest of the system, (d) a series of optical elements for conveying the excitation light to the specimen, collecting the emitted fluorescence in a spatially resolved fashion, and forming an image from this fluorescence emission (or another type of intensity map relevant to the method of detection and measurement), (e) a bench or stand which holds the container of the cells being measured in a predetermined geometry with respect to the series of optical elements, (f) a detector to record the light intensity, preferably in the form of an image, (g) a computer or electronic system and associated software to acquire and store the recorded information and/or images, and to compute the degree of redistribution from the recorded Images.

In a preferred embodiment of the invention the apparatus system is automated. In one embodiment the components mentioned in (d) and (e) above comprise a fluorescence microscope. In one embodiment the component mentioned in (f) above is a CCD camera. In one embodiment the component mentioned in (f) above is an array of photomultiplier tubes/devices.

In one embodiment of the invention the actual fluorescence measurements are made in a standard type of fluorometer for plates of microtiter type (fluorescence plate reader).

In one embodiment an optical scanning system is used to illuminate the bottom of a plate of microtiter type so that a time-resolved recording of changes in luminescence or fluorescence can be made from all spatial limitations simultaneously.

In one embodiment the image is formed and recorded by an optical scanning system.

In a preferred embodiment the actual luminescence or fluorescence measurements are made in a FLIPR™ instrument, commercially available from Molecular Devices, Inc.

The quantitative information which is indicative of the degree of the cellular response to the influence or the result of the influence on the intracellular pathway is extracted from the recording or recordings according to a predetermined calibration based on responses or results, recorded in the same manner; to known degrees of a relevant specific influence. Hereby the degree of redistribution caused by an influence is expressed as the dose of a relevant specific influence causing same degree of cellular response. By testing a unknown influence, e.g. new chemical entities or chemicals without known effect on the redistribution of the cellular component, a screening assay for drugs with effect on redistribution is achieved.

In one embodiment of the invention the screening program is used for the identification of a biologically toxic substance that exerts its toxic effect by interfering with an intracellular signalling pathway. Based on measurements in living cells of the redistribution of spatially resolved luminescence from luminophores which undergo a change in distribution upon activation or deactivation of an intracellular signalling pathway the result of the individual measurement of each substance being screened indicates its potential biologically toxic activity. In one embodiment of a screening program a compound that modulates a component of an intracellular pathway as defined herein, can be found and the therapeutic amount of the compound estimated by a method according to the method of the invention. In a preferred embodiment the present invention leads to the discovery of a new way of treating a condition or disease related to the intracellular function of a biologically active polypeptide comprising administration to a patient suffering from said condition or disease of an effective amount of a compound which has been discovered by any method according to the invention. In another preferred embodiment of the invention a method is established for identification of a new drug target or several new drug targets among the group of biologically active polypeptides which are components of intracellular signalling pathways. This aspect of the invention is described in detail in patent application WO99/23615.

It is preferred that the luminophore incorporates a fluorescent protein such as the fluorophore GFP. Using fluorescence detection methods the distribution of GFP can be visualised continuously. In the present context, the term “green fluorescent protein” (GFP) is intended to indicate a protein which, when expressed by a cell, emits fluorescence upon exposure to light of the correct excitation wavelength (e.g. as described by Chalfie, M. et al. (1994) Science 263, 802-805). Such a fluorescent protein in which one or more amino acids have been substituted, inserted or deleted is also termed “GFP”. “GFP” as used herein includes wild-type GFP derived from the jellyfish Aequorea victona, or from other members of the Coelenterata, such as the red fluorescent protein from Discosoma sp. (Matz, M. V. et al. 1999, Nature Biotechnology 17: 969-973) or fluorescent proteins from other animals, fungi or plants, and modifications of GFP, such as the blue fluorescent variant of GFP disclosed by Heim et al. (Heim, R. et al., 1994, Proc.Natl.Acad.Sci. 91:26, pp 12501-12504), and other modifications that change the spectral properties of the GFP fluorescence, or modifications that exhibit increased fluorescence when expressed in cells at a temperature above about 30° C. described in PCT/DK96/00051, published as WO 97/11094 on Mar. 27, 1997 and hereby incorporated by reference, and which comprises a fluorescent protein derived from Aequorea Green Fluorescent Protein or any functional analogue thereof, wherein the amino acid in position 1 upstream from the chromophore has been mutated to provide an increase of fluorescence intensity when the fluorescent protein of the invention is expressed in cells. Preferred GFP variants are F64L-GFP, F64L-Y66H-GFP F64L-S65T-GFP, F64L-E222G-GFP. One especially preferred variant of GFP for use in all the aspects of this invention is EGFP (DNA encoding EGFP which is a F64L-S65T variant with codons optimized for expression in mammalian cells is available from Clontech, Palo Alto, plasmids containing the EGFP DNA sequence, cf. GenBank Acc. Nos. U55762, U55763). Another especially preferred variant of GFP is F64L-E222G-GFP.

The sensitivity of this aspect of the invention has been markedly improved by the present invention. Now, the signal change resulting from a GFP-tagged protein that redistributes within cells, as measured on the FLIPR™ (Fluorometric Imaging Plate Reader) instrument, can be enhanced several-fold without a concomitant increase in the variability (noise) of the measurement. Furthermore, due to the increase in assay signal, it is now likely that the redistribution assays can be performed in new and denser screening formats, such as 384- and 1536-well microtiter plates in e.g. the FLIPR™ instrument, thereby greatly improving throughput. The redistribution assays may even be compatible with the format described in WO99/35496.

After probing several other chemical compounds for a similar enhancing effect the compound Acid Red 88 (CAS No. 1658-56-6) was identified (see FIGS. 2-4). Acid Red 88 is water soluble but more lipophilic than Trypan Blue and probably enters the cells to some extent and in a concentration-dependent manner. This conclusion is based on a) experiments where Acid Red 88 reduces overall basal fluorescence in a type 1 redistribution assay in a concentration dependent fashion (see FIG. 7) while only marginally increasing the cell-free plate background under similar conditions (see FIG. 8) and b) a reduction and inversion of the type 2 response with increasing concentration of Acid Red 88 (see example 3). The dramatic magnification of the signal from type 1 and 3 redistribution assays using Acid Red 88 in the extracellular medium makes this the enhancer compound of choice for screen development and actual drug screening in these two types of assays.

It can easily be understood that if a compound with GFP fluorescence-reducing properties like those presented here for Acid Red 88 and for Trypan Blue could be directed to a specific cellular compartment, such as the cell nucleus, the endoplasmic reticulum, lysosomes or mitochondria, the compound could be used to specifically reduce the fluorescence from, in that compartment, aggregating GFP-tagged protein. Thereby, redistribution type assays hitherto only possible to perform on an imaging system like a microscope or an ArrayScan™ could be made on fluorescence plate readers such as for example the FLIPR™ instrument. Examples of these types of redistribution assays are those where the GFP- or fluorophore-tagged protein moves from the nucleus to the cytosol, from the cytosol to the nucleus, from or to mitochondria, lysosomes or endoplasmic reticulum.

It is also possible that, with the addition to the liquid volume around the cells of Acid Red 88 (or any other enhancer compound with similar chemical and physical properties that enhance the assay response), sufficiently large light intensity changes will be obtained in any type 1 or type 3 redistribution assay upon redistribution of the fluorescently labelled protein that any state-of-the-art fluorescence plate reader may be used to perform the measurement.

One possible explanation for the effects of Trypan Blue and Acid Red 88 in enhancing type 1 and 3 redistribution assays is that they are efficient absorbers of light emitted from excited GFP. This is important for two reasons. First, in order for a compound to function as a bulk reducer of light emitted by GFP, this spectral overlap must be present. Second, the spectral overlap implies that there is an approximate energy equivalence between the excited state to ground state transition of the GFP and the ground state to excited state transition of the enhancer. If the enhancer can be brought sufficiently close in space to the GFP, a non-radiative energy transfer process can take place between the two molecules which would reduce the fluorescence of the GFP.

The table below shows the average molar absorptivity in the 500-550 nm range, and at 488 nm, for the three dyes Orange G, Acid Red 88, and Trypan Blue. The values were estimated from published spectra of the three substances (Green, FJ, The Sigma-Aldrich Handbook of Stains, Dyes, and Indicators, 1990, Sigma-Aldrich).

| | | Molar | Molar |
|---|---|---|---|
| | | Absorptivity | Absorptivity |
| | Wavelength of maxiumum | 500-550 nm | 488 nm |
| Compound | absorbance (nm) | (mol⁻¹-cm⁻¹) | (mol⁻¹-cm⁻¹) |
| Orange G | 475 | 4524 | 13571 |
| Acid Red 88 | 505 | 14964 | 16986 |
| Trypan Blue | 607 | 34556 | 16014 |

Clearly, the difference in molar absorptivity in the 500-550 nm range is much larger than the difference at 488 nm. The difference at 488 nm is not sufficient to explain the difference in enhancer effect of the three dyes. Trypan Blue, even though its absorption maximum is well outside the 500-550 range, has the largest molar absorptivity in the range of GFP emission. Thus one would expect it to have the largest enhancer effect of the three dyes in assays where this mechanism of enhancement prevails. The weak effect of Orange G in type 1 and 3 redistribution assays further teaches that the average molar absorptivity in the 500-550 range needs to be at least greater than that of Orange G, or approximately 5000 mol⁻¹-cm⁻¹.

Thus, it is preferred that the enhancer compound has an absorption spectrum overlapping the emission spectrum of the luminophore. If the enhancer compound is In itself fluorescent, a narrow bandpass filter should be used. When GFP is the luminophore in question, it is preferred that the enhancer compound absorbs light to a measurable degree in the range of 500-550 nm. It is not necessary that the peak absorbance is within this wavelength range. However, it is important that the average molar absorptivity within this wavelength range is more than 5,000 mol⁻¹-cm⁻¹, such as more than 10,000 mol⁻¹-cm⁻¹.

Another possible explanation for the effects of Trypan Blue and Acid Red 88 in enhancing type 1 and 3 redistribution assays is that the property is dependent on the distance between the GFP molecules emitting the light and the absorbing molecules of the compound. In addition to that, there may be a difference between Trypan Blue and Acid Red 88 in that the latter can penetrate more easily into the cells in a concentration dependent manner and thereby reduce the GFP fluorescence more effectively than Trypan Blue close to the membrane. There is experimental evidence for this; a) In a type 1 redistribution response the enhancing effect of Acid Red 88 first displays an increase at low concentrations and thereafter, compared to the maximal enhancement (see FIG. 3), a decrease at higher concentrations. b) Compared to Trypan Blue, that does not penetrate into cells and therefore does not interfere with a type 2 redistribution response (see example 3), Acid Red 88 at a low concentration removed the type 2 response and at higher concentrations inverted the signal to become a decrease in fluorescence rather than the expected increase (see FIG. 9). This could be interpreted so that at a low concentration enough Acid Red 88 is in the cytoplasm to reduce the fluorescence from redistributing cPKA-GFP (the type 2 redistribution response in example 3) dispersed in the cytosol to a non-detectable fluorescence change. At higher concentrations almost all the fluorescence from redistributing cPKA-GFP dispersed in the cytosol is attenuated by Acid Red 88 but the aggregated form of cPKA-GFP is not affected. This is corroborated by the fact that Acid Red 88 did not reduce the overall basal fluorescence in a type 2 redistribution assay but rather increased it in a concentration dependent fashion (see FIG. 10), wherease in the type 1 redistribution assay it did reduce the basal fluorescence. Thus, it is preferred that the enhancer compound enters the cell, or the cell membrane. The capability of an enhancer compound to enter the cell, or the cell membrane, is generally considered to be related to the solubility of the compound in a lipid-like environment (such as a non-polar organic solvent) relative to the solubility of the compound in the cytoplasm (aqueous solution). This property is conveniently represented by the logP value (the common logarithm of P, the partition coefficent, which is the ratio of the concentration of the compound in octanol and in water, where the octanol and water have been in contact long enough for the compound to equilibrate between them. Based on the knowledge gained from decades of experience in the pharmaceutical industry with regard to the physicochemical properties of compounds which have and have not become drugs, the optimal logP value for compounds capable of entering the cell is less than five, preferably in the range of 1-5.

Another way of evaluate the capability of an enhancer compound to enter the cell or cell membrane is to test the ability to improve the response in a type 2 and a type 1 or 3 redistribution assay respectively.

In order to identify compounds with a similar effect to the known enhancer compounds, the following set of experiments can be performed:

An absorption spectrum should be obtained or measured for the compound to be tested and should display a sufficiently high average molar extinction coefficient for light in the wavelength range 500-550 nm where GFP has its emission peak (see FIG. 5 for an example). An alternative method is to mix the compound to be tested at different concentrations with a fixed concentration of GFP in a cuvette and measure whether the emitted fluorescence from GFP is reduced with increasing concentration of the compound.

Determine if the compound to be tested is sufficiently soluble in aqueous solution so that it can be used in experimental buffer at an appropriate concentration.

Determine the degree of lipophilicity of the compound or test in another way the degree of penetration of the compound into the cells

Test the compound against reference enhancer compounds like Trypan Blue and Acid Red 88 in a type 1, 2 and/or type 3 redistribution assay with a fixed stimuli for both compound and reference compounds and/or

Test the compound against reference compounds like Acid Red 88 in a type 1 and in a type 2 redistribution assay with a fixed stimuli for both compound and reference compounds using a range of concentrations of the tested compounds to look for a peak enhancement effect in the type 1 redistribution assay and a reversal of the signal change in the type 2 redistribution assay.

Any compound that is similar or better than the reference enhancer compounds in any respect of these tests should be considered a enhancer compound. Obviously, such enhancer compound should be tested more thoroughly with regard to enhancement of the redistribution signal in type 1, 2 and 3 assays.

In its broadest aspect, the present invention relates to enhancer compounds with properties that irrespective of chemical or physical mechanism enhance the signal for a redistribution assay where the movement of a fluorescently tagged intracellular protein is monitored in a fluorescence plate reader of any type and detectable as a change in light intensity.

In a preferred aspect of the invention the plate reader has a sensitivity and a small signal detection limit comparable to that of the FLIPR™ instrument from Molecular Devices. It is further preferred that the instrument is equipped to do experiments in 96-, 384- or 1536- well microtiter plate format.

In one aspect of the invention the enhancer compounds are used to enhance the signal for a redistribution assay monitored in a fluorescence microscope as a change in average light intensity over the entire detection area of the detector.

The preferred enhancer compounds are those that absorb light within the visible spectrum and thereby are coloured to the eye when in solution. Even though Trypan Blue has a relatively low solubility in water (<0,1 mg/ml), the solubility is high enough to colour the solution. It should be noted that the solubility in water may be different from the solubility in the aqueous solution, e.g. buffer, used for the experiment. Typically the buffer used for experiments with live cells is about pH 7.4.

One type of preferred enhancer compounds is soluble in aqueous solution at concentrations that are useful for enhancing redistribution signals while not being harmful to the cells.

One type of preferred enhancer compounds is soluble in aqueous solution and does not in any way enter the cell.

One type of preferred enhancer compounds is soluble in aqueous solution but does partly distribute to compartments within the cells in a way that enhances redistribution signals.

One type of preferred enhancer compounds is soluble in aqueous solution and does distribute throughout the cells in a way that enhances redistribution signals.

One type of preferred enhancer compounds maximally absorb light within the wavelength range 400-800 nm.

One type of preferred enhancer compounds maximally absorbs light within the wavelength range 450-600 nm.

One type of preferred enhancer compounds maximally absorbs light within the wavelength range 480-570 nm.

One type of preferred enhancer compounds maximally absorbs light within the wavelength range 500-550 nm.

By using a enhancer compound with properties that, irrespective of chemical or physical mechanism, enhance the signal for a redistribution assay the CV for the assay is usually less than five per cent. In a screening assay with a CV of less than five per cent, single-point screening, i.e. only one test per compound screened, can be performed in a drug discovery setting (this allows an assay to be set up such that there is about 99% certainty that an effect is not an experimental artefact).

### BRIEF DESCRIPTION OF THE FIGURES AND DRAWINGS

FIG. 1.

The CHO (Chinese hamster ovary) cell line stably expressing the human isoform of PKC (protein kinase C) beta 1 tagged with GFP (green fluorescent protein)in its C-terminal end and the human RACK1 (receptor for activated C kinase) was cultured in 96-well microtiter plates (Packard View-Plate). After 20 min preincubation in KRW (experimental buffer; A modified Krebs-Ringer buffer containing (in mM): NaCl 140, KCl 3.6, NaH₂PO₄ 0.5, MgSO₄ 0.5, NaHCO₃ 2.0, CaCl₂ 1.5 and HEPES 10, D-glucose 5 (pH 7.4, titrated with 1M NaOH)) the cell plate was supplemented with a concentration gradient of Trypan Blue at 0, 10, 50, 100 and 200 μM (4 wells per compound and concentration) and placed in the FLIPR™. Subsequently the cells were stimulated in the FLIPR™ with 100 μM ATP. This gives a Type 1 redistribution response (see FIG. 6) in these cells where the redistribution can be quantified as a reduction in fluorescence intensity, as PKC beta 1 moves from the cytosol to the membrane, during the first 1-2 minutes of stimulation. The figure shows 5 traces, each the average of four individual experimental traces. All data were corrected for background by subtraction of a background trace based on 16 individual background traces and normalized to 1 at the point of addition of 100 μM ATP.

FIG. 2.

The CHO (Chinese hamster ovary) cell line stably expressing the human isoform of PKC (protein kinase C) beta 1 tagged with GFP (green fluorescent protein)in its C-terminal end and the human RACK1 (receptor for activated C kinase) was cultured in 96-well microtiter plates (Packard View-Plate). After 20 min preincubation in KRW (experimental buffer; A modified Krebs-Ringer buffer containing (in mM): NaCl 140, KCl 3.6, NaH₂PO₄ 0.5, MgSO₄ 0.5, NaHCO₃ 2.0, CaCl₂ 1.5 and HEPES 10, D-glucose 5 (pH 7.4, titrated with 1M NaOH)) the cell plate was supplemented with a concentration gradient of Acid Red 88 at 0, 10, 50, 100 and 200 μM (4 wells per compound and concentration) and placed in the FLIPR™. Subsequently the cells were stimulated in the FLIPR™ with 100 μM ATP. This gives a Type 1 redistribution response (see FIG. 6) in these cells where the redistribution can be quantified as a reduction in fluorescence intensity, as PKC beta 1 moves from the cytosol to the membrane, during the first 1-2 minutes of stimulation. The figure shows 5 traces, each the average of four individual experimental traces. All data were corrected for background by subtraction of a background trace based on 16 individual background traces and normalized to 1 at the point of addition of 100 μM ATP.

FIG. 3.

The CHO (Chinese hamster ovary) cell line stably expressing the human isoform of PKC (protein kinase C) beta 1 tagged with GFP (green fluorescent protein) in its C-terminal end and the human RACK1 (receptor for activated C kinase) was cultured in 96-well microtiter plates (Packard View-Plate). After 20 min preincubation in KRW (experimental buffer; A modified Krebs-Ringer buffer containing (in mM): NaCl 140, KCl 3.6, NaH₂PO₄ 0.5, MgSO₄ 0.5, NaHCO₃ 2.0, CaCl₂ 1.5 and HEPES 10, D-glucose 5 (pH 7.4, titrated with 1M NaOH)) the cell plate was supplemented with a concentration gradient of Trypan Blue, Acid Red 88 and Orange G at 0, 10, 50, 100 and 200 μM (4 wells per compound and concentration) and placed in the FLIPR™. Subsequently the cells were stimulated in the FLIPR™ with 100 μM ATP. This gives a Type 1 redistribution response (see FIG. 6) in these cells where the redistribution can be quantified as a reduction in fluorescence intensity, as PKC beta 1 moves from the cytosol to the membrane, during the first 1-2 minutes of stimulation. All data were corrected for background by subtraction of a background trace based on 16 individual background traces and normalized to 1 at the point of addition of 100 μM ATP. The data points represent the average of four determinations+SD. The data were extracted as the maximal change (SMax-SMin) during the first minute of stimulation and all positive values despite the fact that the actual responses are reductions in fluorescence intensity (Smax is the highest fluorescence value during the time frame and Smin is the lowest value. Since the response was negative the double minus sign in the end gives a positive value). The zero value for concentration depicts the presence of just experimental buffer.

FIG. 4.

The CHO (Chinese hamster ovary) cell line stably expressing the PH-domain (pleckstrin homology) of the human isoform of PLC (phospho-lipase C) delta tagged with GFP (green fluorescent protein) in its C-terminal end was cultured in 96-well microtiter plates (Packard View-Plate). After 20 min preincubation in KRW (experimental buffer; A modified Krebs-Ringer buffer containing (in mM): NaCl 140, KCl 3.6, NaH₂PO₄ 0.5, MgSO₄ 0.5, NaHCO₃ 2.0, CaCl₂ 1.5 and HEPES 10, D-glucose 5 (pH 7.4, titrated with 1M NaOH)) the cell plate was supplemented with 100 μM Trypan Blue in one half and 10 μM Acid Red 88 in the other half of the wells and placed in the FLIPR™. Subsequently the cells were stimulated in the FLIPR™ with a range of ATP concentrations starting with 300 μM and lower by a threefold reduction of concentration in each dilution step. This gives a Type 3 redistribution response (see FIG. 6) in these cells where the redistribution can be quantified as an increase in fluorescence intensity, as the PH-domain of PLC delta 1 moves from the membrane to the cytosol, during the first 1-2 minutes of stimulation. FIG. 4 summarizes this experiment. All data were corrected for background by subtraction of a background trace based on 4 individual background traces. The data points represent the average of four determinations+SD. The data were extracted as the maximal change during the first minute of stimulation (Smax-Smin).

FIG. 5.

Light absorption spectra for the redistribution signal enhancers Trypan Blue, Acid Red 88 and Orange G. The absorption values are given as molar extinction coefficient. It can be noted that Trypan Blue has the highest absorption capacity per mole in the wavelength range 500-550 nm and that Acid Red 88 has its absorption maximum within the same range whereas both Orange G and Trypan Blue has their peaks outside this range.

FIG. 6.

Schematic representation of different types of redistribution responses alluded to in the text.

Type 1 is representative for instance for the initial stages of PKC beta 1 redistribution in response to a receptor stimulus like ATP when expressed tagged with GFP In CHO cells.

Type 2 is representative for instance for the initial stages of cPKA-GFP redistribution In response to a stimulation of the activity of the intracellular enzyme Adenylatecyclase to produce cAMP with the compound Forskolin when the cPKA-GFP construct is expressed in CHO cells.

Type 3 is representative for instance for the initial stages of PLCdeltaPH-domain-GFP redistribution in response to a receptor stimulus like ATP when the construct is expressed in CHO cells.

FIG. 7.

The CHO (Chinese hamster ovary) cell line stably expressing the human isoform of PKC (protein kinase C) beta 1 tagged with GFP (green fluorescent protein) in its C-terminal end and the human RACK1 (receptor for activated C kinase) was cultured in 96-well microtiter plates (Packard View-Plate). After 20 min preincubation in KRW (experimental buffer; A modified Krebs-Ringer buffer containing (in mM): NaCl 140, KCl 3.6, NaH₂PO₄ 0.5, MgSO₄ 0.5, NaHCO₃ 2.0, CaCl₂ 1.5 and HEPES 10, D-glucose 5 (pH 7.4, titrated with 1M NaOH)) the cell plate was supplemented with a concentration gradient of Trypan Blue and Acid Red 88 at 0, 10, 50, 100 and 200 μM during the experimental fluorescence recording (4 wells per compound and concentration) in the FLIPR™. All data were corrected for background by subtraction of a background trace based on 16 individual background traces and normalized to 1 at the point of addition of the various concentrations of Trypan Blue and Acid Red 88. The data points represent the average of four determinations+SD. The data were extracted as the absolute fluorescence change two minutes after addition of the two compounds. The zero value displays the reduction in overall fluorescence from a control addition of just experimental buffer.

FIG. 8.

Experiments were made in 96-well microtiter plates (Packard View-Plate) without any cells, just containing KRW (experimental buffer; A modified Krebs-Ringer buffer containing (in mM): NaCl 140, KCl 3.6, NaH₂PO₄ 0.5, MgSO₄ 0.5, NaHCO₃ 2.0, CaCl₂ 1.5 and HEPES 10, D-glucose 5 (pH 7.4, titrated with 1M NaOH)). The cell plate was supplemented with a concentration gradient of Trypan Blue and Acid Red 88 at 0, 10, 50, 100 and 200 μM during the experimental fluorescence recording (4 wells per compound and concentration) in the FLIPR™. All data were corrected for background by subtraction of a background trace based on 16 individual background traces and normalized to 1 at the point of addition of the various concentrations of Trypan Blue and Acid Red 88. The data points represent the average of four determinations+SD. The data were extracted as the absolute fluorescence change 30 seconds after addition of the two compounds. The zero value displays the increase in overall fluorescence from a control addition of just experimental buffer.

FIG. 9.

The CHO (Chinese hamster ovary) cell line stably expressing the mouse cPKA (catalytic subunit of protein kinase A) tagged with GFP (green fluorescent protein) in its N-terminal end was cultured in 96-well microtiter plates (Packard View-Plate). After 20 min preincubation in KRW (experimental buffer; A modified Krebs-Ringer buffer containing (in mM): NaCl 140, KCl 3.6, NaH₂PO₄ 0.5, MgSO₄ 0.5, NaHCO₃ 2.0, CaCl₂ 1.5 and HEPES 10, D-glucose 5 (pH 7.4, titrated with 1M NaOH)) the cell plate was supplemented with a concentration gradient of Trypan Blue and Acid Red 88 (4 wells per compound and concentration) and placed in the FLIPR™. Subsequently the cells were stimulated in the FLIPR™ with 10 μM Forskolin. This gives a Type 2 redistribution response (see FIG. 6) in these cells where the redistribution can be quantified as an increase in fluorescence intensity, as cPKA moves from aggregates in the cytosol to a dispersed form in the cytosol, during the first 2-5 minutes of stimulation. All data were corrected for background by subtraction of a background trace based on 16 individual background traces and normalized to 1 at the point of addition of 10 μM Forskolin. The data points represent the average of four determinations+SD. The data were extracted as the maximal change in fluorescence during the first five minutes of stimulation. Negative values depict a decrease In fluorescence during the response and positive values and Increase. The zero value displays the increase in overall fluorescence from an addition of 10 μM Forskolin in the presence of just experimental buffer.

FIG. 10.

The CHO (Chinese hamster ovary) cell line stably expressing the mouse cPKA (catalytic subunit of protein kinase A) tagged with GFP (green fluorescent protein) in its N-terminal end was cultured in 96-well microtiter plates (Packard View-Plate). After 20 min preincubation in KRW (experimental buffer; A modified Krebs-Ringer buffer containing (in mM): NaCl 140, KCl 3.6, NaH₂PO₄ 0.5, MgSO₄ 0.5, NaHCO₃ 2.0, CaCl₂ 1.5 and HEPES 10, D-glucose 5 (pH 7.4, titrated with 1M NaOH)) the cell plate was supplemented with a concentration gradient of Trypan Blue and Acid Red 88 at 0, 10, 50, 100 and 200 μM during the experimental fluorescence recording (4 wells per compound and concentration) in the FLIPR™. All data were corrected for background by subtraction of a background trace based on 16 individual background traces and normalized to 1 at the point of addition of the various concentrations of Trypan Blue and Acid Red 88. The data points represent the average of four determinations+SD. The data were extracted as the absolute fluorescence change 30 seconds after addition of the two compounds. The zero value displays the decrease in overall fluorescence from a control addition of just experimental buffer.

FIG. 11a:

Insulin-stimulated translocation of hGLUT4-EGFP to plasma membrane quenched by Acid Red-88. A 96-well plate of 3T3-L1 adipocytes are stimulated with insulin 30 min before measuring hGLUT4-EGFP translocation to the plasma membrane. The effect of Acid Red-88 on EGFP fluoresence is evaluated in a time course experiment spanning from 0-1260 seconds with measurements each 6 seconds. Acid Red-88 (10μM) is added at 60 seconds. Insulin concentrations are 100 nM, 10 nM, 1 nM, 0.10 nM, 0.01 nM, and 0 nM as indicated in the figure. Each time point is an average of 8 Identically treated wells. Values are normalized to the curve obtained for 0 nM insulin and set to zero at 54 seconds.

FIG. 11b:

Magnification of curves in FIG. 11a before addition of Acid Red-88 (0-54 seconds).

FIG. 12:

Dose-response curve of GLUT4 redistribution after Insulin stimulation. Sum of fluorescence intensity measurements from 60 seconds to 600 seconds belonging to each insulin concentration. Values are normalized to measurements without addition of insulin (0 nM Ins) and set to zero at 54 seconds.

### EXAMPLES

### Example 1

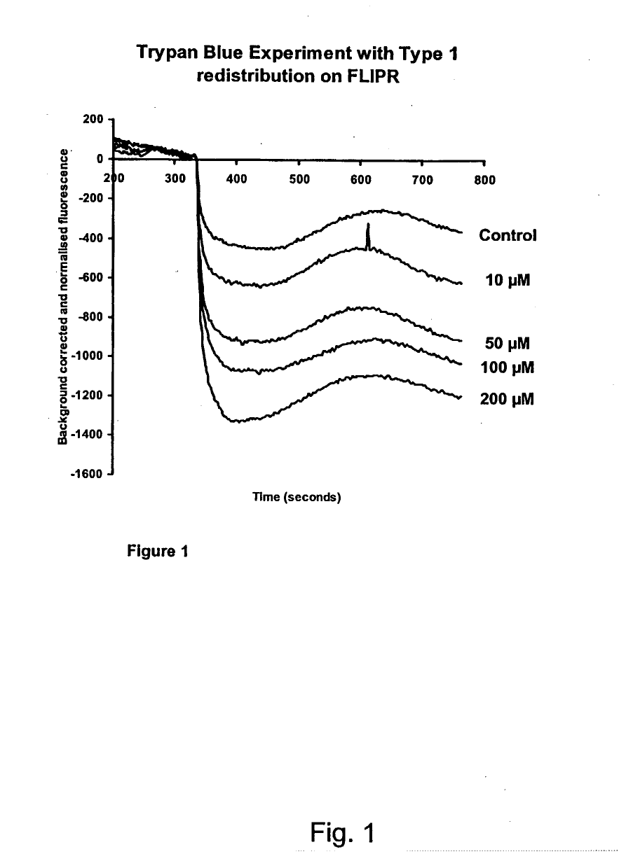

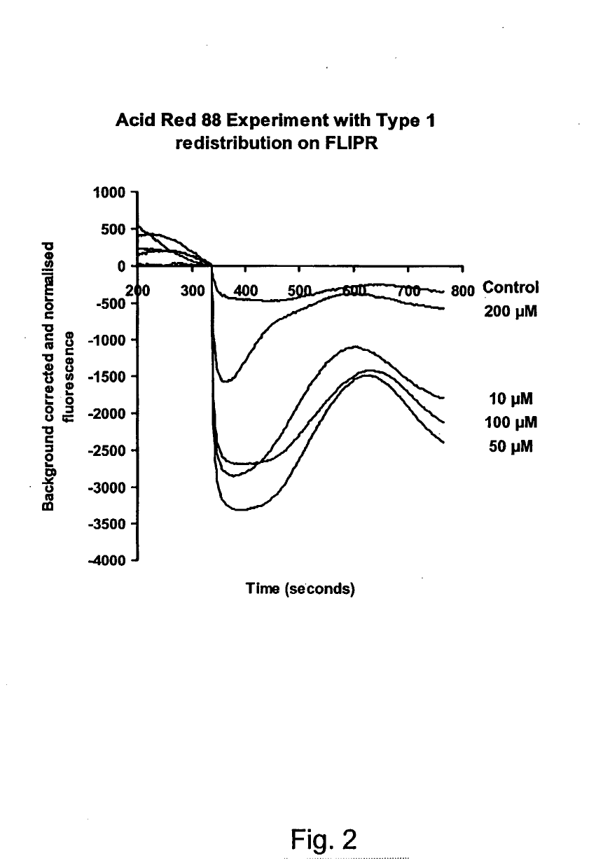

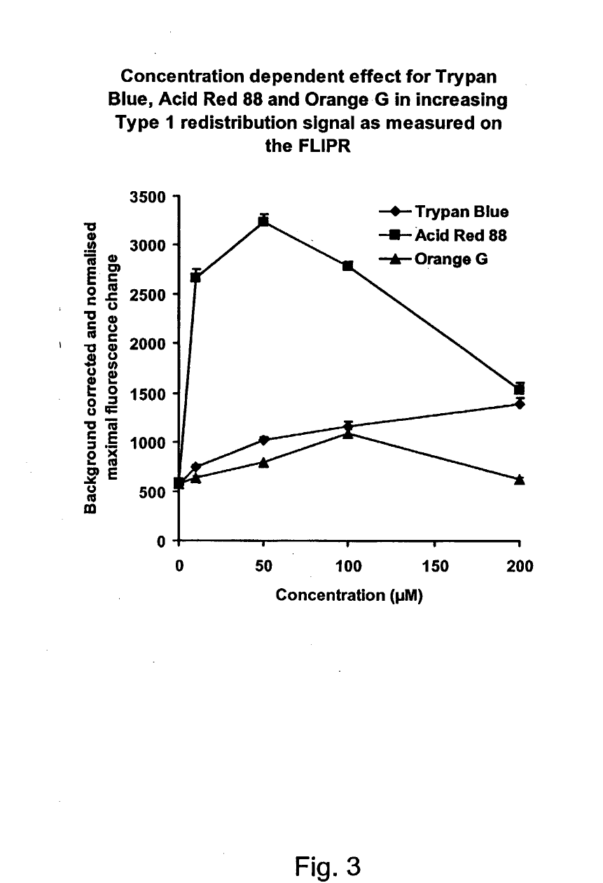

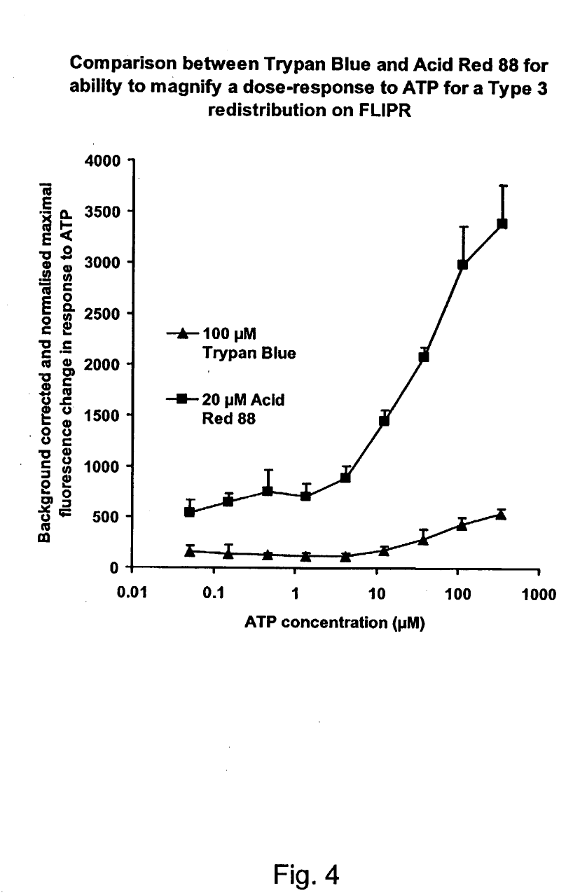

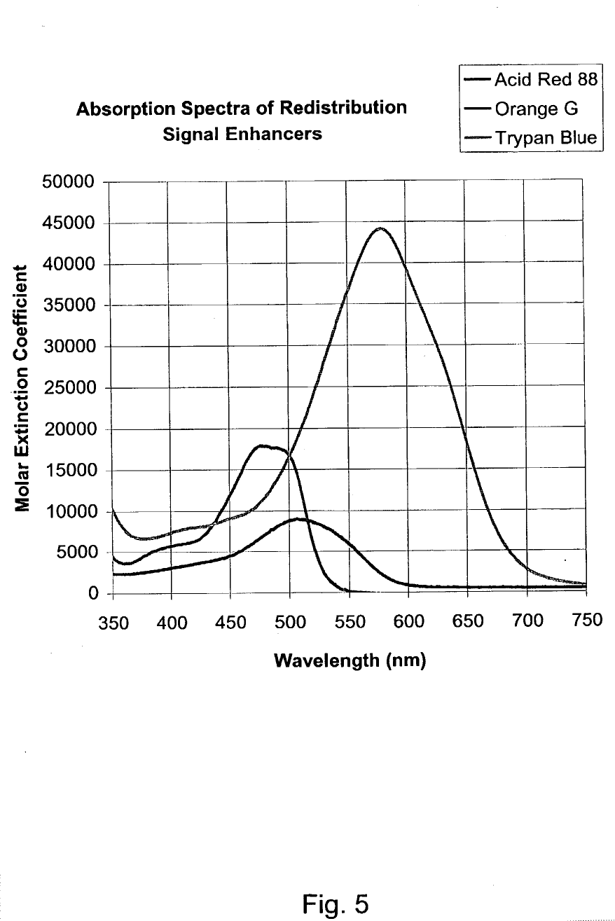

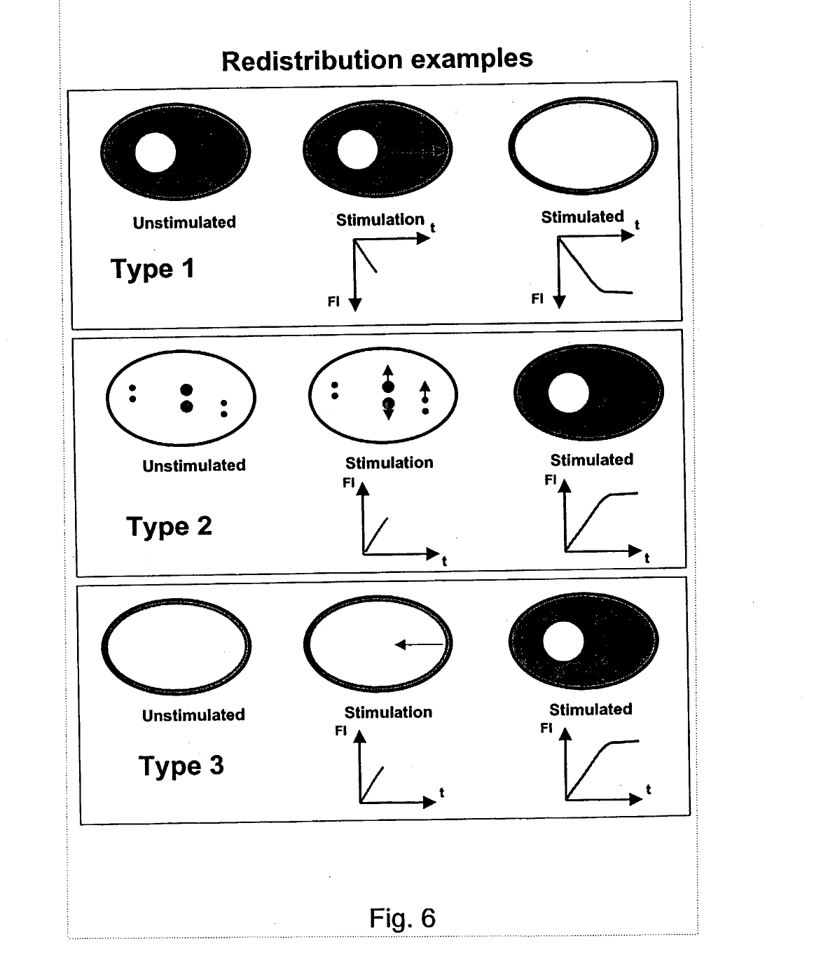

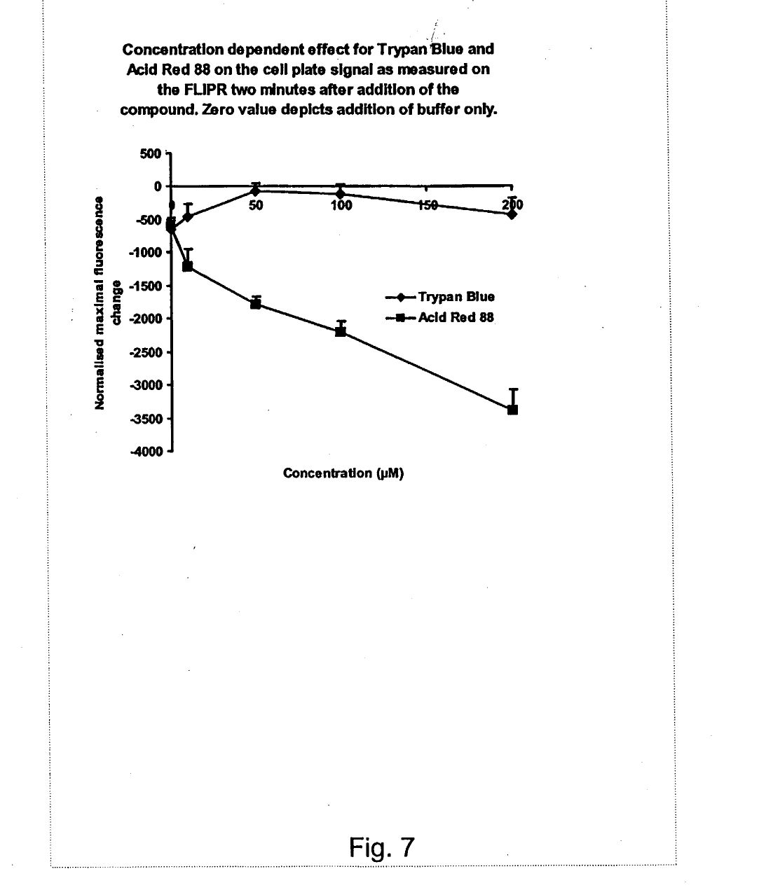

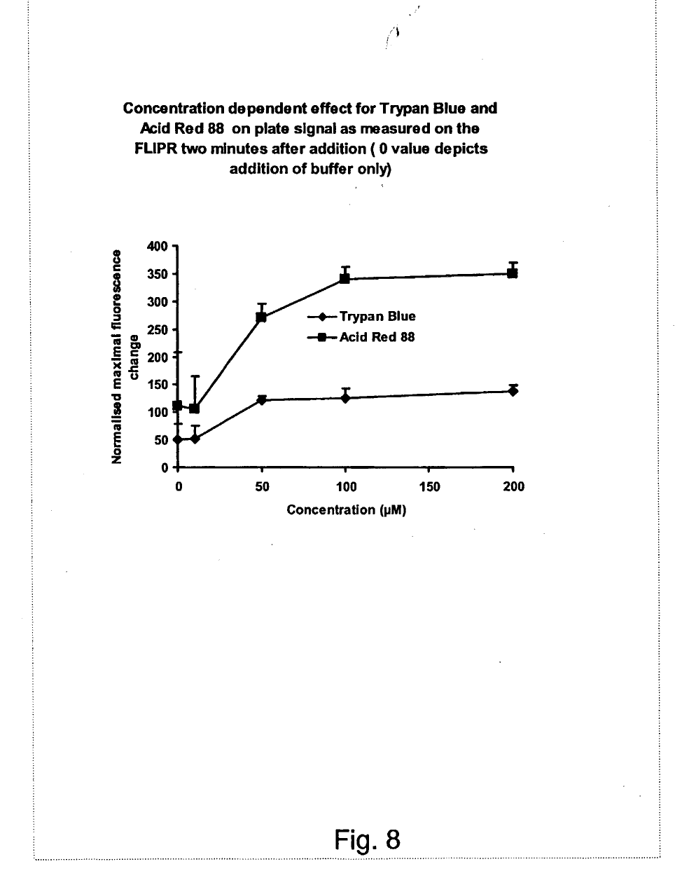

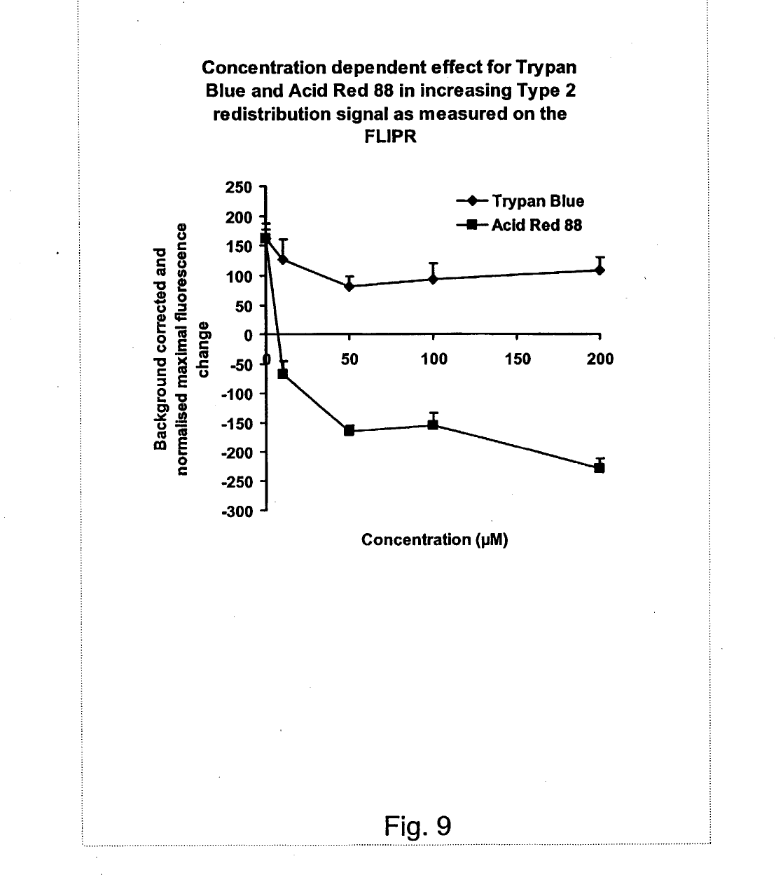

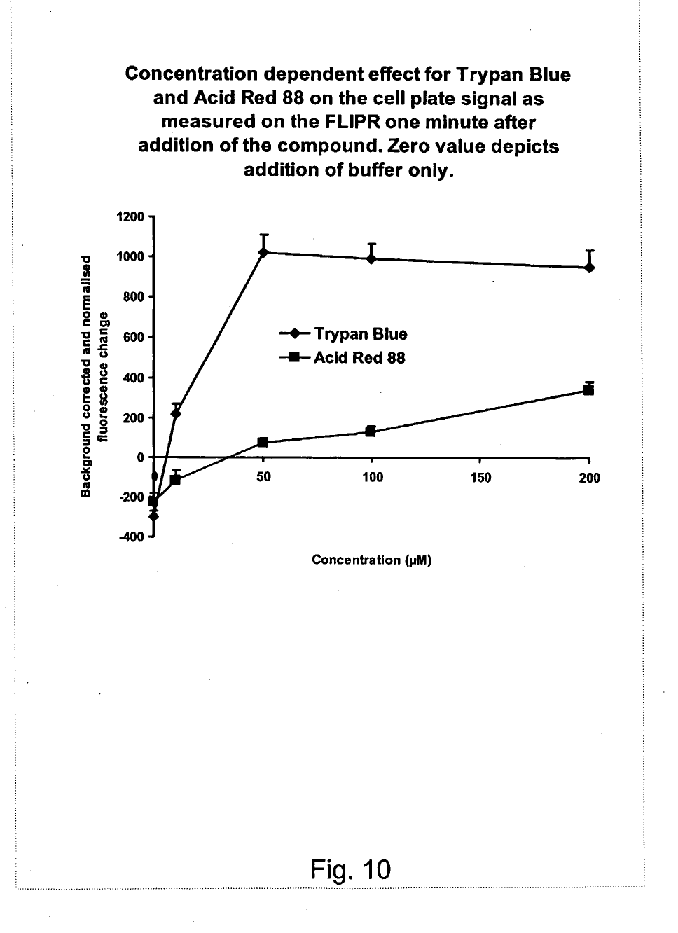

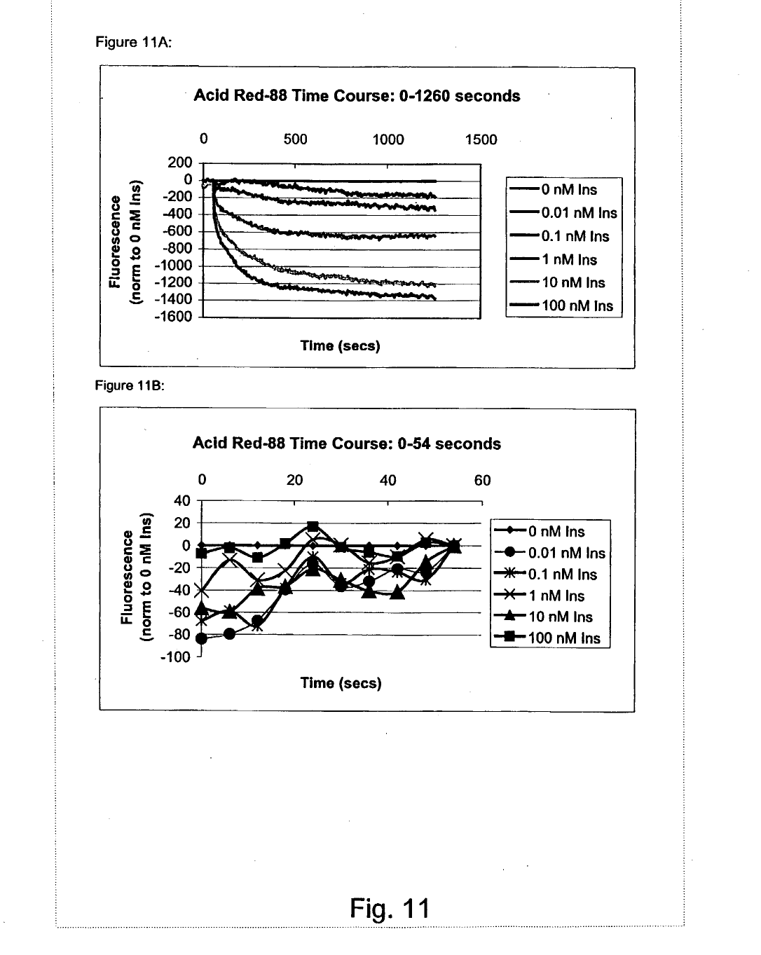

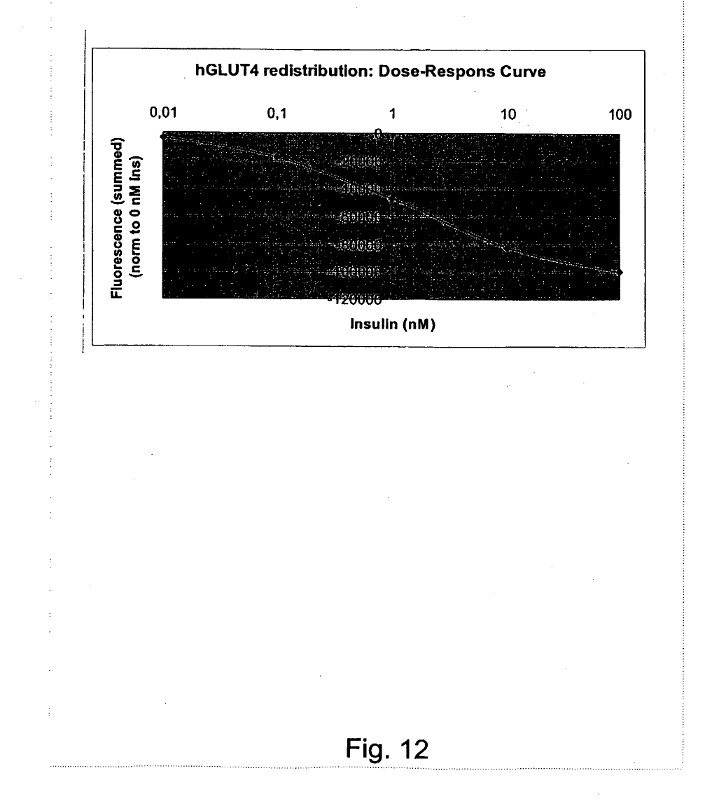

The CHO (Chinese hamster ovary) cell line stably expressing the human isoform of PKC (protein kinase C) beta 1 tagged with GFP (green fluorescent protein) in its C-terminal end and also expressing the human RACK1 (receptor for activated C kinase) was cultured in 96-well microtiter plates (Packard View-Plate). After 20 min preincubation in KRW (experimental buffer; A modified Krebs-Ringer buffer containing (in mM): NaCl 140, KCl 3.6, NaH₂PO₄ 0.5, MgSO₄ 0.5, NaHCO₃ 2.0, CaCl₂ 1.5 and HEPES 10, D-glucose 5 (ph 7.4, titrated with 1 M NaOH)) the cell plate was supplemented with a concentration gradient of Trypan Blue, Acid Red 88 and Orange G (4 wells per compound and concentration) and placed in the FLIPR™. Subsequently the cells were stimulated in the FLIPR™ with 100 μM ATP. This gives a Type 1 redistribution response (see FIG. 6) in these cells where the redistribution can be quantified as a reduction in fluorescence intensity, as PKC beta 1 moves from the cytosol to the membrane, during the first 1-2 minutes of stimulation. FIGS. 1, 2 and 3 are summaries of this experiment. As can be seen In FIGS. 1 and 3, increasing concentrations of Trypan Blue enhanced the response compared to the control in a concentration dependent fashion. The maximal enhancement was about 3-fold at 200 μM. As shown in FIG. 2 and 3, all concentrations of Acid Red 88 enhanced the response compared to the control. However, the maximal enhancement was about 7-fold at 50 μM and higher concentrations of Acid Red 88 displayed smaller levels of enhancement.

Finally, in FIG. 3 it is demonstrated that Orange G also has enhancing properties up to a concentration of 100 μM but is overall less effective that Trypan Blue.

In summary, for type 1 redistribution response measured on the FLIPR™ instrument Trypan Blue added to the extracellular medium gives a 2-3-fold increase in the measurable response without altering its kinetics significantly. Acid Red 88 gives at best a 6-7-fold increase in the measurable response without altering its kinetics but does affect the kinetics at higher than optimal (50 μM in this example) concentrations. An appropriate concentration of Acid Red 88 makes a redistribution assay for type 1 redistribution sensitive enough to do single well screening (i.e. to only do one determination for each test of an unknown compound in a drug screen), thereby maximizing the through-put of the FLIPR™ instrument. Orange G is inferior to Trypan Blue and much inferior to Acid Red 88 in increasing the type I redistribution response measured on the FLIPR™ instrument.

### Example 2

The CHO (Chinese hamster ovary) cell line stably expressing the PH-domain (pleckstrin homology) of the human isoform of PLC (phospho-lipase C) delta tagged with GFP (green fluorescent protein) in its C-terminal end was cultured in 96-well microtiter plates (Packard View-Plate). After 20 min preincubation in KRW (experimental buffer; A modified Krebs-Ringer buffer containing (in mM): NaCl 140, KCl 3.6, NaH₂PO₄ 0.5, MgSO₄ 0.5, NaHCO₃ 2.0, CaCl₂ 1.5 and HEPES 10, D-glucose 5 (pH 7.4, titrated with 1M NaOH)) the cell plate was supplemented with 100 μM Trypan Blue in one half and 20 μM Acid Red 88 in the other half of the wells and placed in the FLIPR. Subsequently the cells were stimulated in the FLIPR with 100 μM ATP. This gives a Type 3 redistribution response (see FIG. 6) in these cells where the redistribution can be quantified as an increase in fluorescence intensity, as the PH-domain of PLC delta 1 moves from the membrane to the cytosol, during the first 1-2 minutes of stimulation. FIG. 4 summarizes this experiment. It can be seen that Acid Red 88 in this case maximally gives about a 6-7-fold increase in signal over the one obtained with Trypan Blue. Trypan Blue does however clearly give a detectable signal for this type 3 redistribution response, which is barely detectable without the compound.

In summary, for type 3 redistribution response measured on the FLIPR instrument Trypan Blue added to the extracellular medium gives about a 1-3-fold increase in the measurable response without altering its kinetics significantly. Acid Red 88 gives at best a 7-10-fold increase in the measurable response without altering its kinetics. An appropriate concentration of Acid Red 88 makes a redistribution assay for type 3 redistribution sensitive enough to do single well screening (i.e. to only do one determination for each test of an unknown compound in a drug screen), thereby maximizing the through-put of the FLIPR instrument.

### Example 3

The CHO (Chinese hamster ovary) cell line stably expressing the mouse cPKA (catalytic subunit of protein kinase A) tagged with GFP (green fluorescent protein) in its N-terminal end was cultured in 96-well microtiter plates (Packard View-Plate). After 20 min preincubation in KRW (experimental buffer; A modified Krebs-Ringer buffer containing (in mM): NaCl 140, KCl 3.6, NaH₂PO₄ 0.5, MgSO₄ 0.5, NaHCO₃ 2.0, CaCl₂ 1.5 and HEPES 10, D-glucose 5 (pH 7.4, titrated with 1M NaOH)) the cell plate was supplemented with a concentration gradient of Trypan Blue, Acid Red 88 and Orange G (4 wells per compound and concentration) and placed in the FLIPR. Subsequently the cells were stimulated in the FLIPR with 10 μM Forskolin. This gives a Type 2 redistribution response (see FIG. 6) in these cells where the redistribution can be quantified as an increase in fluorescence intensity, as cPKA moves from aggregates in the cytosol to a dispersed form in the cytosol, during the first 2-5 minutes of stimulation.

In summary, for type 2 redistribution response measured on the FLIPR instrument neither of the dyes did at all enhance the response over the control level (se FIG. 9). Trypan Blue even reduced it somewhat at higher concentrations. Acid Red 88 completely abolished the response at the lowest concentration and then reversed it to a fluorescence decrease at higher concentrations (above 10 μM) that became larger in magnitude in an almost concentration-dependent fashion.

### Example 4

Here is described a human glucose transporter 4 (hGLUT4) redistribution assay in living cells that takes advantage of Acid Red-88 as an agent that quench GFP fluorescence at the cellular plasma membrane.

Insulin stimulates glucose uptake into adipocyte and muscle cells by inducing the translocation of GLUT4 from an intracellular microsomal compartment to the plasma membrane. This insulin-dependent redistribution event can be studied in cell culture by the use of 3T3-L1 adipocytes. The procedure described below takes advantage of a combination of highly efficient gene delivery by retrovirus, differentiation of hGLUT4-EGFP expressing 3T3L1 fibroblasts to 3T3-L1 adipocytes, and use of Acid Red-88 to monitor hGLUT4-EGFP redistribution to the plasma membrane of living cells. Construction of pBabe-Puro-hGLUT4-EGFP retroviral expression vector A DNA fragment encoding human GLUT4 (GenBank m20747) is amplified by PCR using the primer set 0212: 5′-GCCAAGCTTCTGCCATGCCGTCGGGCTTCCAACAGATAGGCTCC and 0213:5′-GGCGAATTCCGTCGTTCTCATCTGGCCCTAAATACTCAAGTTCTGTGC and human HeLa cell cDNA (Clontech, Palo Alto) as template. This fragment is digested with Hindlll and EcoRI, and ligated into the corresponding sites in pEGFP-N1 (Clontech, Palo Alto; GenBank U55762). An hGLUT4-EGFP fragment is excised from this plasmid using Hindlll and Xbal. The hGLUT4-EGFP fragment is then end-filled with klenow and ligated into SnaBI-digested pBabe-Puro (Morgenstern and Land, Nucleic Acid Research. 1990; 12: 3587-3596).

Stable ΦNX-Eco hGLUT4-EGFP Retrovirus Producer Line

The day before transfection, ΦDNX-Eco cells (Phoenix-Eco, 293T packaging cell line producing high titres of retrovirus) are plated to 50% confluency in a 25 cm2 flask in Dulbecco's modified Eagle's medium (GibcoBRL/Life Technologies, Rockville, Cat #10566) supplemented with 10% fetal calf serum, and 100 units/ml penicillin and streptomycin in a humidified atmosphere at 37° C. containing 5% CO₂.ΦNX-Eco cells are transfected with pBabe-Puro-hGLUT4-EGFP using Lipofectamine 2000 transfection reagent according to the manufacturer's instructions (Life Technologies, Rockville). Stable ΦNX-Eco hGLUT4-EGFP retrovirus producer cell are selected and propagated in growth medium containing 2 μg/ml puromycin (Sigma, St. Louis).

Transduction and Differentiation of 3T3-L1 Fibroblasts

Low passage 3T3-L1 fibroblasts (passage <20) are plated at 50% confluency in 75 cm² flasks in Dulbecco's modified Eagle's medium (GibcoBRL/Life Technologies, Rockville, Cat #10566) supplemented with 10% calf serum, and 100 units/ml penicillin and streptomycin in a humidified atmosphere at 37° C. containing 5% CO₂. The following day, the medium is replaced by diluted supernatant from confluent ΦNX-Eco hGLUT4-EGFP retrovirus producer cells (dilution 1:1 with fresh 3T3-L1 growth medium) supplemented with hexadimethrin bromide (polybrene) (Sigma, St. Louis) to a final concentration of 6 μg/ml. The following day, retroviruses have integrated the hGLUT4-EGFP expression cassette into the 3T3-L1 host genome and hGLUT4-EGFP expression can be detected in transduced cells. At this stage the 3T3-L1 fibroblasts are ready for differentiation into adipocytes. Note that 3T3-L1 fibroblasts must never be allowed to reach more than 70% confluence in order for them to retain their ability to fully differentiate. The ratio of cells that differentiate in a successful differentiation amounts to 80-90%.

The differentiation procedure runs over 10 days starting at day −2 and ending at day +7 relative to day 0, the day of addition of differentiation inducers. For differentiation, transduced 3T3-L1 fibroblasts are plated at 50-100% confluency in 96-well plates in Dulbecco's modified Eagle's medium (GibcoBRL/Life Technologies, Rockville, Cat #10566) supplemented with 10% calf serum, and 100 units/ml penicillin and streptomycin.

Day−2; The cells are allowed to reach confluence (Day−2).

Day 0: The medium is replaced by Dulbecco's modified Eagle's medium (GibcoBRL/Life Technologies, Rockville, Cat #10566) supplemented with 10% fetal calf serum, 1 μM dexamethasone (Sigma, St. Louis), 0.5 mM 3-isobutyl-1-methylxanthine (Sigma, St. Louis), 167 nM human insulin (Sigma, St. Louis), and 100 units/ml penicillin and streptomycin.

Day +2: The medium is replaced by Dulbecco's modified Eagle's medium (GibcoBRL/Life Technologies, Rockville, Cat #10566) supplemented with 10% fetal calf serum, 167 nM human insulin (Sigma, St. Louis), and 100 units/ml penicillin and streptomycin.

Day +4: The medium is replaced by Dulbecco's modified Eagle's medium (GibcoBRL/Life Technologies, Rockville, Cat #10566) supplemented with 10% fetal calf serum and 100 units/ml penicillin and streptomycin. The following days the medium is changed every other day. The 3T3-L1 adipocytes are fully differentiated at day +7.

GLUT4 Redistribution Assay

Transduced 3T3-L1 fibroblasts expressing hGLUT4-EGFP are differentiated to adipocytes in Packard 96-well ViewPlates as decribed above and assayed 8 days after induction of differentiation (day +8). Immediately prior to running the assay, the adipocytes are starved for 2 hours at 37° C. in starvation medium, Dulbecco's modified Eagle's medium (GibcoBRL/Life Technologies, Rockville, Cat #10566) supplemented with 0.1% BSA. Then, the adipocytes are equilibrated for 30 min at 37° C. in assay buffer, Krebs-Ringer-Wollheim (KRW) buffer pH 7.4 (140 mM NaCl, 2 mM NaHCO₃, 3.6 mM KCl, 0.5 mM NaH₂PO₄, 0.5 mM MgSO₄, 1 mM CaCl₂, 10 mM Hepes supplemented with 0.1% BSA and 25 mM glucose. Insulin diluted in KRW buffer is added ending at a total volume of 180 μl KRW buffer per well. Cells are incubated 30 min at 37° C. and transferred to FLIPR preheated to 37° C. The FLIPR is set to 1 W with 6 seconds between each measurement ending at 1260 seconds (21 min). After 60 seconds, volumes of 20 μl 100 μM Acid Red-88 are added to each well In FLIPR (final concentration 10 μM Acid Red-88 ).

Results and Discussion:

Insulin stimulates glucose uptake into adipocytes by inducing the translocation of the glucose transporter 4 (GLUT4) from an intracellular microsomal compartment to the plasma membrane. In order to monitor this redistribution event in living cells, human GLUT4 (hGLUT4) is fused to the N-terminus of enhanced green fluorescent protein (EGFP) and inserted into the retroviral vector pBabe-Puro. Infectious retroviruses containing the hGLUT4-EGFP cassette is produced in 293T ΦNX-Eco packaging cells and used to deliver hGLUT4-EGFP to 3T3-L1 fibroblasts. In this way, a population of 3T3-L1 fibroblasts are made that express hGLUT4-EGFP stably. These cells are plated in 96-well plates and differentiated into hGLUT4-EGFP expressing 3T3-L1 adipocytes as described above.

To measure hGLUT4-EGFP redistribution, living 3T3-L1 hGLUT4-EGFP adipocytes are starved for 2 hours in serum-free medium in order to retain hGLUT4-EGFP in its basal localization in microsomal compartments proximal to the cell nucleus. From this location, redistribution of a fraction of hGLUT4-EGFP to the plasma membrane can be induced by insulin stimulation. Immediately, following starvation and equilibration in KRW assay buffer, insulin is added to 8-well duplicates in concentrations varying from 0-100 nM human insulin. Redistribution is allowed to proceed at 37° C. for 30 min before EGFP fluorescence is measured in the FLIPR preheated to 37° C. The FLIPR is set to measure every 6 seconds in a time course experiment stopping at 1260 seconds. The level of hGLUT4-EGFP at the plasma membrane is estimated by quenching the EGFP fluorescence at the membrane by addition of 10 μM Acid Red-88 60 seconds after the first measurement. In FIG. 1a, EGFP fluorescence over time is illustrated for each insulin concentration normalized to fluorescence from the basal state localization (0 nM insulin). Without addition of Acid-Red (0-54 seconds), differences in detected fluorescence between different doses of insulin are not discernable (FIG. 11b). Addition of Acid Red-88 (at 60 seconds) results in quenching of EGFP fluorescence from the plasma membrane and thereby in reduction of total fluorescence from adipocytes in which hGLUT4-EGFP has redistributed to the plasma membrane (FIG. 11a). Following this procedure, it is possible to calculate a dose-response curve for insulin-dependent redistribution of hGLUT4-EGFP to the plasma membrane of 3T3-L1 adipocytes. Integration of the area below the individual curves in FIG. 1a between 60-600 seconds results in the dose-response curve shown in FIG. 12. The ED50 for this assay can be calculated to ED50=1 nM insulin.

1. A method for measuring redistribution of a luminophore coupled to an intracellular component of a cell comprising
(a) adding an enhancer compound to the cell/cell medium and
(b) measuring changes in light intensity.

2. A method according to claim 1, wherein the enhancer compound is soluble in aqueous solution.

3. A method according to any of the preceding claims, wherein the enhancer compound has an absorption spectrum overlapping the emission spectrum of the luminophore.

4. A method according to the preceding claim, wherein the absorption spectrum is 500-550 nm and the luminophore is GFP.

5. A method according to any of the preceding claims, wherein the enhancer compound has an average molar absorptivity of more than 5.000 mol⁻¹-cm⁻¹.

6. A method according to any of the preceding claims, wherein the enhancer compound enters the cell, or the cell membrane.

7. A method according to any of the preceding claims, wherein the enhancer compound is localised primarily at a specific cellular compartment, and wherein the redistribution assay is an assay for redistribution to or from said specific cellular compartment.

8. A method according to any of the preceding claims, wherein the enhancer compound is substantially localised in the extracellular medium.

9. A method according to any of the preceding claims, wherein the redistribution is movement from the cytosol to the membrane.

10. A method according to any of the preceding claims, wherein the redistribution is movement from the membrane to the cytosol.

11. A method according to the two previous claims, wherein the membrane is the plasma membrane.

12. A method according to any of the preceding claims, wherein the luminophore is a fluorophore such as GFP.

13. A method according to any of the preceding claims, wherein the redistribution is measured in a live cell assay.

14. A method according to any of the preceding claims, wherein the changes in light intensity are measured with a device made for fluorescence measurements in any form of array.

15. A method according to any of the preceding claims, wherein the changes in light intensity are measured with a device made for fluorescence measurements in microtiter plate format.

16. A method according to any of the preceding claims, wherein the changes in light intensity are measured with a plate reader.

17. A method according to any of the preceding claims, wherein the changes in light intensity are measured with a fluorescence plate reader.

18. A method according to any of the preceding claims, wherein the changes in light intensity are measured with a FLIPR™.

19. A method according to any of the preceding claims, wherein the enhancer compound is Acid Red.

20. A method according to any of the preceding claims, wherein the enhancer compound is Acid Red 88.

21. A method according to any of the preceding claims, wherein the enhancer compound is Trypan Blue.

22. Use of a enhancer compound for the preparation of an assay for the measurement of redistribution of a fluorophore coupled to an intracellular component, the measurement of redistribution comprising measuring changes in light intensity in a live cell assay.

23. Use according to claim 22, wherein the enhancer compound is Acid Red.

24. Use according to claim 22, wherein the enhancer compound is Acid Red 88.

25. Use according to claim 22, wherein the enhancer compound is Trypan Blue.
